(19) United States
(12) Patent Application Publication
(10) Pub. No.: US 2017/0020634 A1
(43) Pub. Date: Jan. 26, 2017

(54) DENTAL IMPLANT SYSTEM

(71) Applicant: Dietmar SONNLEITNER, Salzburg (AT)

(72) Inventor: Dietmar SONNLEITNER, Salzburg (AT)

(21) Appl. No.: 15/285,829

(22) Filed: Oct. 5, 2016

Related U.S. Application Data
(63) Continuation of application No. PCT/AT2015/000016, filed on Feb. 4, 2015.

(30) Foreign Application Priority Data
Apr. 15, 2014 (AT) A 280/2014

(51) Int. Cl. A61C 8/02; A61F 2/28; A61C 8/00

(52) U.S. Cl. CPC A61C 8/0006; A61C 8/0074; A61C 8/008; A61F 2/2846

(57) ABSTRACT

The invention relates to a dental implant system for bone regeneration of a bone defect site of a jaw bone, comprising: an implant to be anchored in the jaw bone and having a threaded hole; a film for covering the bone defect site and the implant; and a fastening screw for the positionally stable fastening of the film relative to the implant. In the assembly position, a screw bolt of the fastening screw projects through the film and can be screwed together with the threaded hole of the implant, a spacing element being arrangeable between the implant and the film and the screw bolt projecting through the spacing element in the assembly position.

The invention concerns a dental implant system for bone regeneration of a bone defect site of a jawbone including an implant which is to be anchored in the jawbone and has a threaded bore, a film for covering over the bone defect site and the implant, and a fixing screw for positionally stable fixing of the film relative to the implant, wherein in the fitted position a screw bolt of the fixing screw projects through the film and can be screwed to the threaded bore in the implant.

Dental implant systems are already known, using membrane technology, to promote bone regeneration of a bone defect site. Such dental implant systems include an implant which is anchored in the region of the bone defect site in the jawbone, a film or membrane which permits bone regeneration and which is stretched over the bone defect site and thus also over the implant and fixed to the jawbone, and a fixing screw whose screw bolt projects through the film upon being fitted in place and upon positionally stable fixing of the film relative to the implant, and is screwed to a threaded bore in the implant. That provides between the film and a surface of the bone defect site, a cavity in which bone material and in the case of natural teeth also the periodontium can subsequently grow. For advantageous bone regeneration the cavity can also contain bone substitute materials, carriers for drugs, growth factors or other substances which protect and promote healing and bone formation. Depending on the respective configuration of the bone defect site, when using the known dental implant systems, when applying the film or membrane, unwanted fold or crater formation can occur, which in turn can lead to an unwanted surface structure in respect of the regenerated jawbone.

Therefore the object of the invention is to provide an improved dental implant system for bone regeneration of a bone defect site of a jawbone, which in particular facilitates application of the film at the bone defect site and which permits an improved surface structure for the regenerated bone material.

According to the invention that object is attained by the features of claim 1. Advantageous configurations of the invention are recited in the appendant claims.

According to the invention it is therefore provided that a spacer element can be arranged between the implant and the film, wherein in the fitted position the screw bolt projects through the spacer element. The provision of a spacer element between an implant head at the end of the implant and the film which is to be placed over the bone defect site makes it possible to compensate for an unwanted difference in level between a first level of the implant head and a second level of an edge of the bone defect site, in a simple fashion, in dependence on the configuration of the bone defect site. In that case the screw bolt projects through the spacer element and can be screwed to a female thread in the threaded bore of the implant. Due to the fact that the screw bolt projects through the spacer element without engagement therewith the spacer element does not rotate with the fixing screw during the positionally stable fixing of the film, whereby the film is also not turned or creased. Thus two substantial advantages can be achieved with the proposed dental implant system. On the one hand, unwanted crater formation in respect of the film can be avoided by virtue of flexible adaptation to given differences in level between the implant head and the edge of the bone defect site, and on the other hand unwanted folding can be avoided in the region of the film in which the screw bolt projects through the film, as the film is not turned or creased when being fixed in its stable position.

In a preferred embodiment it can be provided that the spacer element for passing the screw bolt therethrough has a through bore of a bore diameter larger than an outside diameter of the screw bolt. In that way the screw bolt projects through the spacer element without making contact therewith and is not in engagement with the spacer element during screwing to the threaded bore in the implant. That already represents a simple way of preventing the spacer element from turning.

A particularly preferred embodiment provides that the spacer element can be arranged on the implant in rotationally locked relationship. Preferably in that respect it can be provided that provided as rotational locking means on the spacer element are projections which can be inserted into corresponding recesses of the implant. A rotationally locked arrangement of the spacer element on the implant ensures that the spacer element is not also rotated with the fixing screw during fixing of the film relative to the implant, that is to say while the fixing screw is being screwed to the implant. It is thus possible to provide a rotation-preventing means for preventing unwanted folding when the film is being fixed.

A particularly advantageous embodiment of the invention is that in which the spacer element is of a substantially hollow cylindrical shape, preferably in the form of a substantially tubular sleeve. In that case the spacer element can be in the form of a spacer sleeve which is particularly easy to manufacture.

It can also be provided that the spacer element is substantially conical or frustoconical. In that way in particular an enlarged contact surface for the film on the spacer element can be formed and the anatomical shape of a natural root can be simulated.

In a preferred embodiment it can be provided that the fixing screw has a screw head, wherein the film can be clamped between the spacer element and the screw head. In that case the screw head of the fixing screw can form a so-called healing post which serves as a place holder for a passage post which is to be fitted after bone regeneration and which keeps open a passage through the gum into the oral cavity.

In order further to reduce folding of the film it can preferably be provided that at a screw head surface which in the fitted position is towards the film the screw head is provided with a friction-reducing coating, preferably with a Teflon coating. It can naturally also be provided that the film is provided with a friction-reducing coating, preferably with a Teflon coating, at a film surface that is towards the screw head in the fitted position, at least in a contact region with the screw head.

In order also to permit a flexible configuration in respect of the height of the healing post to be fitted in accordance with a further preferred embodiment it can be provided that the dental implant system includes a substantially tubular fixing sleeve through which the screw bolt projects in the fitted position, wherein the film can be clamped between the spacer element and the fixing sleeve. In that case the fixing sleeve can form a healing post, whereby various heights of healing posts can be provided in a simple fashion.

Preferably in that respect it can be provided that the fixing sleeve for passing the screw bolt therethrough has a through hole of a hole diameter which is larger than an outside diameter of the screw bolt. In that case the screw bolt projects without contact through the fixing sleeve whereby the fixing sleeve is not also rotated with the fixing screw during fixing of the screw and thus no unwanted turning or folding of the film takes place.

A particular variant provides that provided on the fixing sleeve at a sleeve surface which in the fitted position is towards the film is at least one extension—preferably of a substantially thorn-like configuration—for positional fixing of the film relative to the fixing sleeve. That can prevent the film from turning or creasing.

In a particularly preferred embodiment it can be provided that the film is substantially completely resorbable. Because the film or membrane overall is completely resorbable in the body by being broken down for example by hydrolysis in the body there is no need to perform a further operation for removal of the film after bone regeneration has occurred.

A particularly advantageous embodiment is that in which the film is a preferably pre-bonded multi-layer film which includes a shaping forming layer for shaping the film to the bone defect site and at least one cover layer for covering the bone defect site, wherein the forming layer and the at least one cover layer are substantially completely resorbable. In that case the film includes a shaping forming layer which serves for shaping the film to the bone defect site and by which a cavity can be formed between the bone defect site and the film so that bone growth can occur in that cavity. The cavity is maintained by the space-forming and space-maintaining forming layer until the cavity is filled up by growing bone material. In addition the film of this embodiment includes at least one cover layer for covering the bone defect site. That cover layer which for example can be in the form of a membrane serves for covering and sealing off the bone defect site to prevent soft tissue from passing into the bone defect site. In order further to improve fitting of the film and the sealing of the bone defect site the at least one cover layer can also be such that it adheres to a gum surrounding the bone defect site. The individual layers of such a pre-bonded film (forming layer and at least one cover layer) can be mechanically and/or chemically bonded together.

In a preferred embodiment it can be provided that the forming layer and the at least one cover layer are substantially completely resorbable in different periods of time. For example, the design of the forming layer and the at least one cover layer makes it possible to provide that the forming layer is resorbed more quickly than the at least one cover layer. Generally, differing degrees of resorbability of the forming layer and the at least one cover layer give great degrees of freedom in the design of the film in relation to resorbability.

It can be provided that the film can be substantially completely resorbed in a period of between about 3 and 12 months, preferably between about 4 and 6 months. That is the period of time within which bone regeneration occurs in the normal case.

To permit good shaping to the bone defect site and stable cavity formation between the film and the bone defect site it can be provided that the forming layer is stiffer than the at least one cover layer. The higher degree of stiffness of the forming layer serves to form a cavity for bone construction and also to maintain that cavity for the period required for bone regeneration. Once again, good coverage and sealing of the bone defect site can be achieved by the at least one cover layer being of lesser stiffness in comparison with the forming layer.

Preferably it can be provided that the forming layer, possibly together with the at least one cover layer, is adapted to be mechanically and/or thermally and/or chemically deformable. Thus in particular the forming layer can be in the form of a layer which is substantially stable in shape and which can be deformed under a mechanical, thermal or chemical influence and which after such deformation again enjoys adequate stability in respect of shape to maintain the cavity to be formed for bone growth, for the required period of time. The at least one cover layer can be flexible and preferably elastic to permit good coverage and sealing of the bone defect site.

In that case mechanical deformation can be effected for example by bending with a tongs. That is a suitable shaping method in particular for comparatively thin forming layers (for example in the range of between about 0.10 mm and about 0.5 mm). For thicker forming layers (for example thicker than about 0.5 mm) thermal deformation of the layer can be appropriate for the shaping operation. Suitable thermal deformation can be achieved in that case for example by means of a thermal bar with a hot tip or surface, by way of heated prefabricated models or in a hot water bath with a sterile saline solution.

For good resorbability of the proposed film it can be provided that the at least one cover layer at least partially and preferably substantially completely comprises a bioresorbable collagen material. In that case it can be provided that the bioresorbable collagen material includes type-I-collagen and/or type-III-collagen. The collagen material can for example originate from bovine Achilles tendon.

For good resorbability of the proposed film it can also be provided that the forming layer at least partially and preferably substantially completely comprises a bioresorbable polymer material. The bioresorbable polymer material can also be a copolymer material.

A particular variant provides that the bioresorbable polymer material includes lactic acid, preferably L-lactic acid, and/or derivatives thereof. It is advantageous in that case if the proportion of lactic acid in the bioresorbable polymer material is at least 70%, preferably between about 80% and 95%, particularly preferably substantially about 82%.

In addition it can be provided that the bioresorbable polymer material includes glycolic acid. It is advantageous in that respect if the proportion of glycolic acid in the bioresorbable polymer material is at most 30%, preferably between about 15% and 20%, particularly preferably substantially about 18%. Depending on the respective composition of the forming layer it can be provided that the forming layer is substantially stable in respect of shape and is nonetheless substantially completely resorbable.

In a further preferred embodiment it can be provided that the forming layer and the at least one cover layer are of different surface areas. In that respect it can be provided that the forming layer is of a smaller surface area than the at least one cover layer. If the at least one cover layer covers over the forming layer by virtue of its smaller surface area it is possible to achieve particularly good coverage and thus also sealing of the bone defect site.

Preferably it can be provided that the at least one cover layer and/or the forming layer is or are of a substantially flat configuration throughout. A contour for the film, which is advantageous for shaping to the bone defect site, can be achieved for example by suitably cutting the film.

It is particularly advantageous however if the forming layer for shaping to the bone defect site has a shape structure. In that case it can be provided that the shape structure has at least portion-wise a convexly and/or concavely curved edge and/or at least portion-wise a convexly and/or concavely curved shape. In other words the shape structure can have for example areal—convexly and/or concavely curved—projections and thus a convexly and/or concavely curved edge. Alternatively or additionally the shape structure as a whole can also be of a correspondingly convexly and/or concavely curved shape.

It is particularly advantageous if the shape structure has at least one strut-shaped shaping element. The strut-shaped or tab-shaped shaping elements can be shaped in a hoop-like configuration over the bone defect site and permit any cavity shape to be produced.

A particularly advantageous embodiment of the invention is that in which the shape structure is of a substantially grid-shaped configuration. The grid-shaped structure in that case forms a reinforcing grid which permits the formation of a plurality of any desired cavity shapes.

It can also be provided that the shape structure is formed by at least one reinforcement of the forming layer. Particularly if the forming layer is applied in the form of a hardening liquid or a hardening gel to the at least one cover layer it is desirable if the shape structure can be achieved merely by applying more liquid or gel in the region of the shape structure. In that case for example the shape structure can be of differing thicknesses.

A particular variant provides that the film has a carrier layer for at least one substance which is arranged or which is to be arranged thereon. The substances arranged or to be arranged on the carrier layer can be drugs, growth factors or other substances for protecting and promoting healing and bone formation. The carrier layer can preferably be arranged at a side of the film, that is to face towards the bone defect site, and can at least partially and preferably substantially completely comprise a bioresorbable collagen material.

It can also be provided that appropriate substances are applied directly to the forming layer and/or the at least one cover layer. It can also be provided that the side or surface of the film, that is to face towards a bone defect site, itself serves as a carrier for the above-described substances, by for example that side or surface of the film having a suitable roughness.

Depending on the respective situation of use the film or membrane can also be provided in pre-cut and/or pre-shaped fashion. In that case for example a desired cut configuration and/or a desired 3D deformation of the film can be effected in accordance with data processing-aided planning.

In general the individual components of the proposed dental implant system, depending on the respective situation of use, can be planned and produced, for example milled, individually in a data processing-aided procedure.

Further details and advantages of the present invention are described hereinafter by means of the specific description. In the drawing:

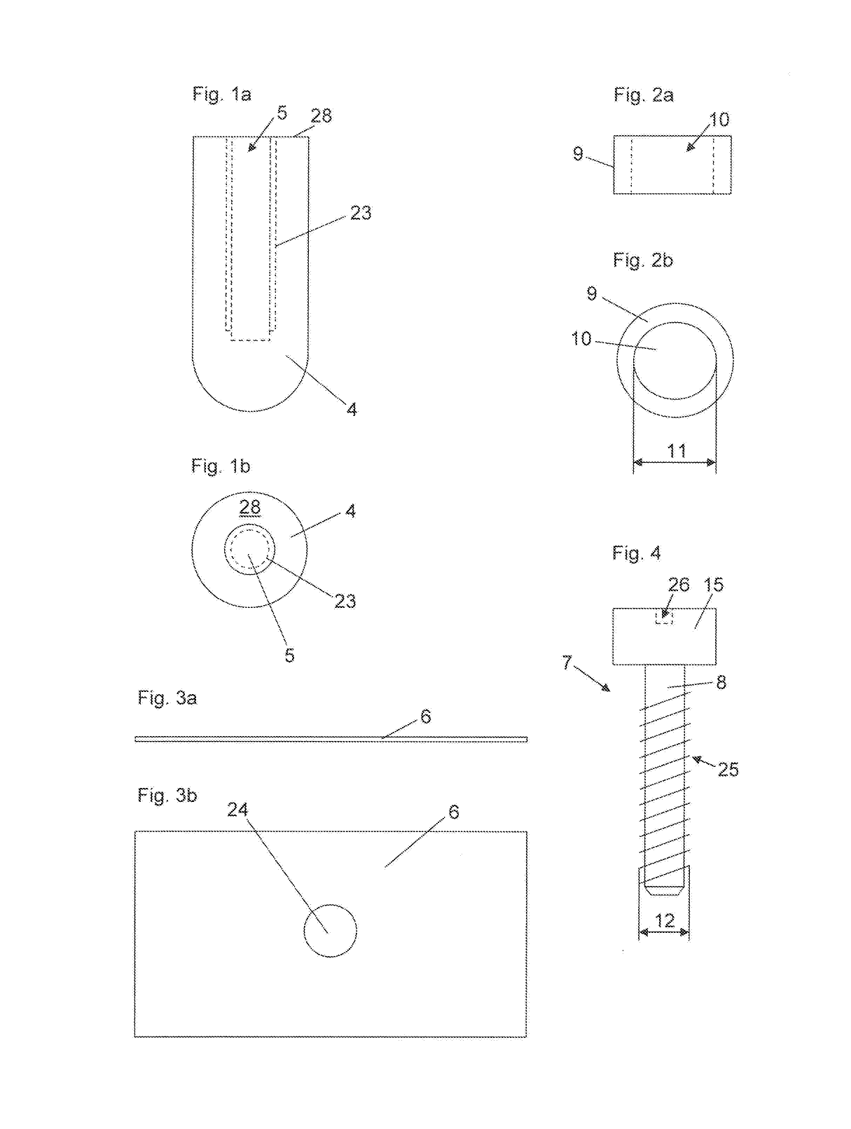

FIG. 1a shows a side view of an implant,

FIG. 1b shows a plan view of the implant of FIG. 1a,

FIG. 2a shows a side view of the proposed spacer element in the form of a spacer sleeve,

FIG. 2b shows a plan view of the spacer element of FIG. 2a,

FIG. 3a shows a side view of a film for covering over a bone defect site and an implant,

FIG. 3b shows a plan view of the film of FIG. 3a,

FIG. 4 shows a side view of a fixing screw,

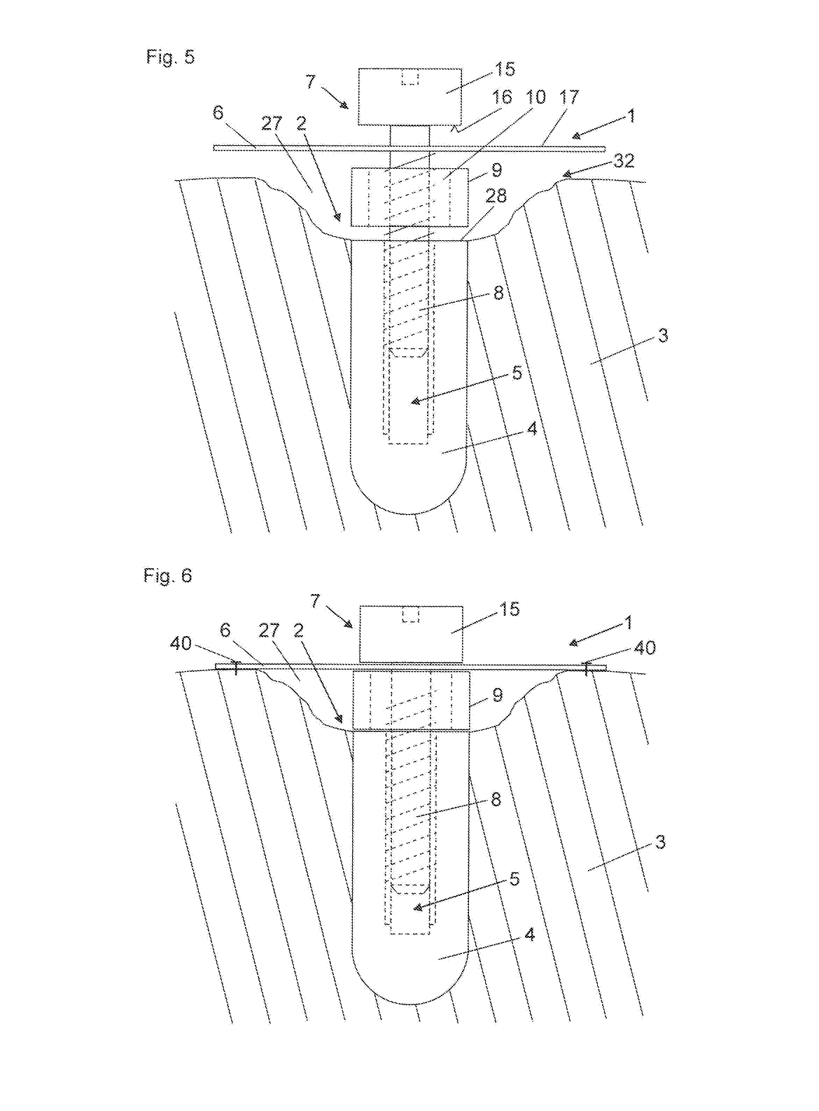

FIG. 5 shows an embodiment of the proposed dental implant system during fixing at a bone defect site of a jawbone,

FIGS. 6-8 show a number of embodiments of the proposed dental implant system in each case disposed at a bone defect site of a jawbone,

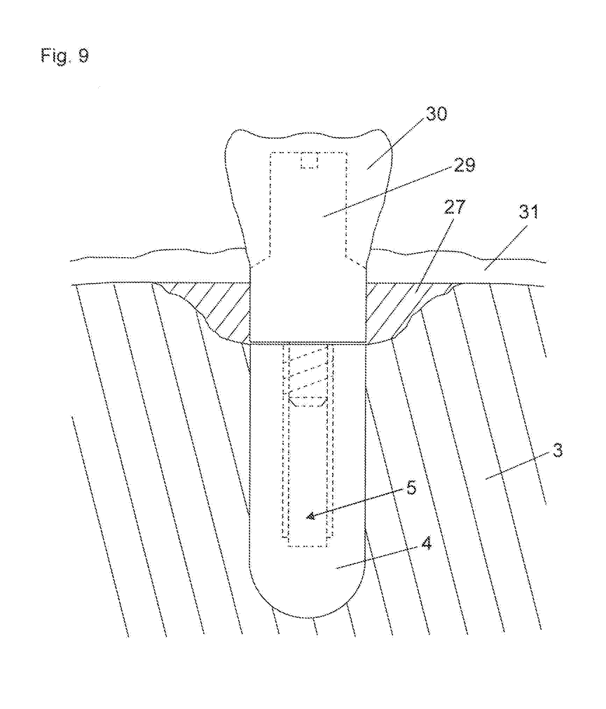

FIG. 9 shows a sectional view through a regenerated bone defect site,

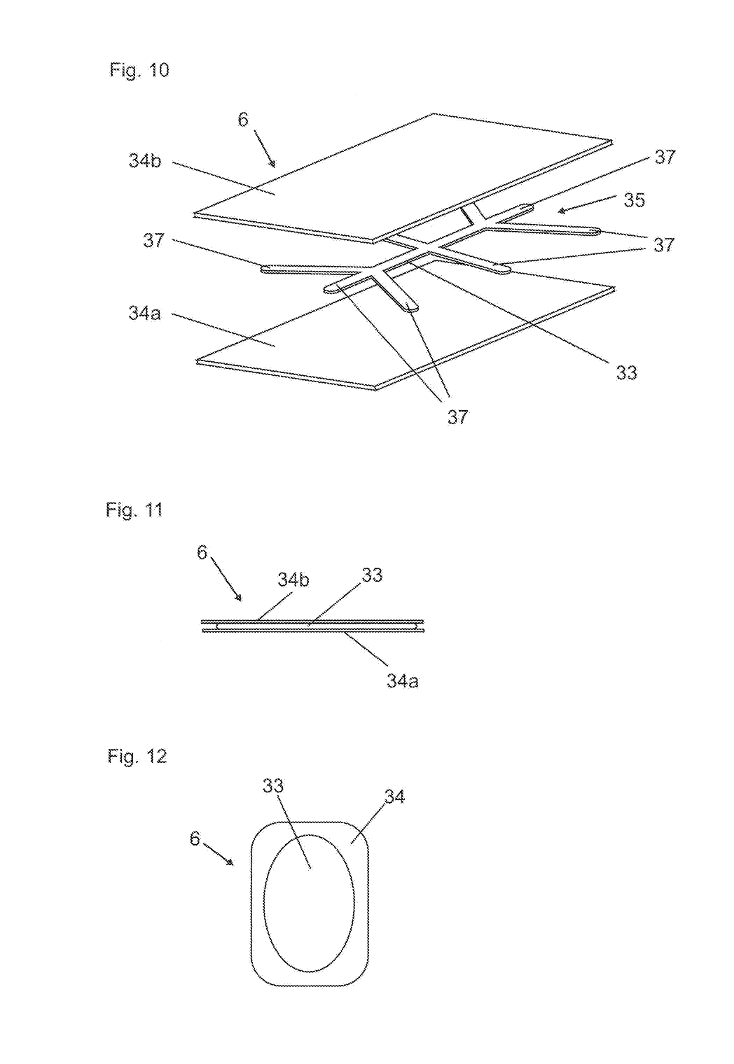

FIG. 10 shows an exploded perspective view of an embodiment of a multi-layer film,

FIG. 11 shows a side view of the multi-layer film of FIG. 10,

FIGS. 12-16 show plan views of various embodiments of multi-layer films, and

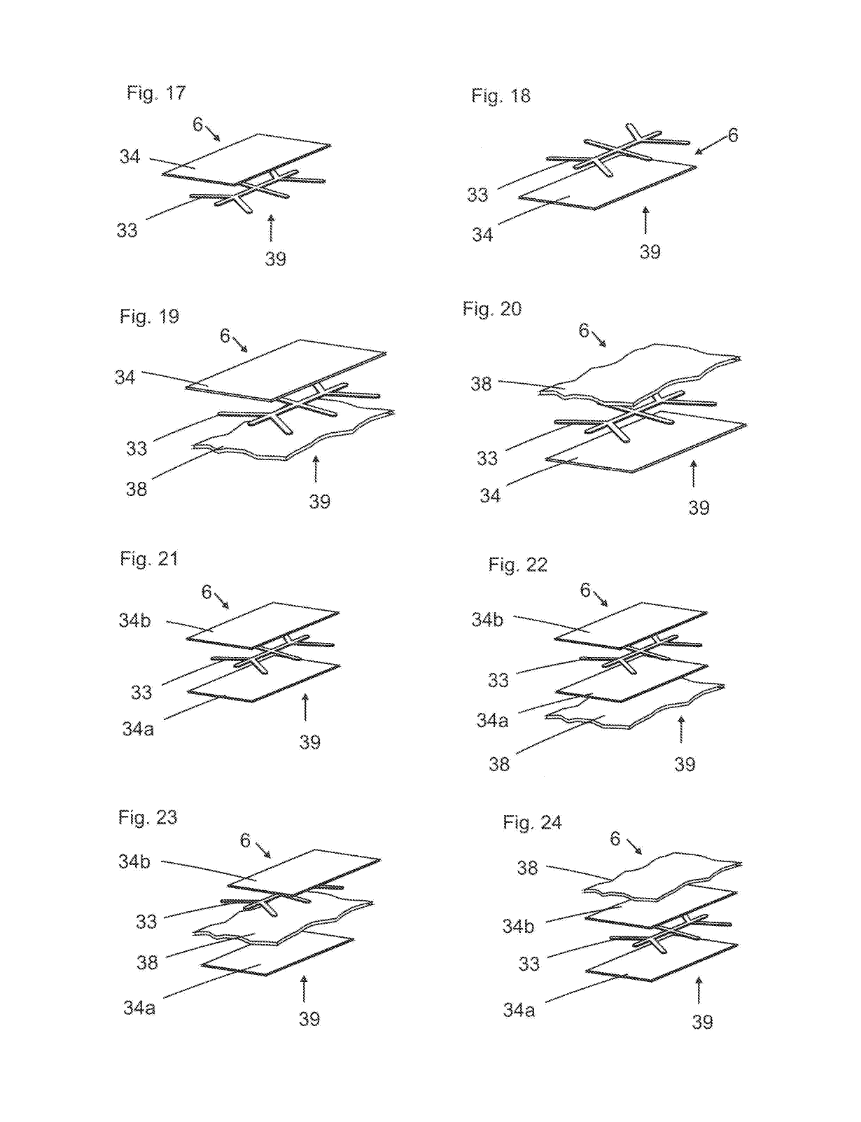

FIGS. 17-24 show exploded perspective views of a number of embodiments of multi-layer films.

FIG. 1a shows a side view of an implant 4 of a proposed dental implant system 1 and FIG. 1b shows a plan view of the implant 4. Starting from the implant head 28 of the implant 4 the implant 4 has a threaded bore 5 provided with a female thread 23.

FIG. 2a shows a side view of a spacer element 9 of a proposed dental implant system 1 and FIG. 2b shows a plan view of the spacer element 9. In this example the spacer element 9 is in the form of a spacer sleeve and has a through bore 10 of a bore diameter 11.

FIG. 3a shows a side view of a film 6 of a proposed dental implant system 1 and FIG. 3b shows a plan view of the film 6. The film 6 has a hole 24, through which in the fitted position a screw bolt 8 of a fixing screw 7 can be passed.

FIG. 4 shows a side view of a fixing screw 7 of a proposed dental implant system 1. The fixing screw 7 has a screw head 15 and a screw bolt 8 which is arranged thereon and which is provided with a thread 25. The screw bolt 8 with the thread 25 and the threaded bore 5 with the female thread 23 are of such dimensions that the thread 25 of the screw bolt 8 can be screwed to the female thread 23 of the threaded bore 5. The screw bolt 8 is of an outside diameter 12 which is less than the bore diameter 11 of the through bore 10 in the spacer element 9. The screw head 15 has a tool recess 26 into which a suitable tool can engage to screw the fixing screw 7 to the implant 4, like for example a screwdriver or a hexagonal Allen key.

FIG. 5 shows a sectional view through a bone defect site 2 of a jawbone 3, an implant 4 being anchored in the jawbone 3 in the region of the bone defect site 2. To permit bone regeneration in the region of the bone defect site 2 a membrane or film 6 is placed over the bone defect site 2 and thus also over the implant 4 and fixed to the jawbone 3 to form a cavity 27 between the bone defect site 2 and the film 6, in which the jawbone 3 can regenerate. Depending on the respective configuration of a bone defect site 2 and anchorage of the implant 4 however there can be different differences in level between a first level of the implant head 28 of the implant 4 and a second level of an edge 32 of the bone defect site 2. In order to achieve uniform bone growth and a desired surface for the regenerated jawbone 3 in spite of such a difference in level there is provided a spacer element 9 which is arranged between the implant 4 and the film 6 to compensate for precisely that difference in level. For positionally stable fixing of the film 6 the screw bolt 8 of a fixing screw 7 is now passed through the film 6 or a corresponding hole 24 in the film 6 and through the through bore 10 in the spacer element 9 and screwed to the threaded bore 5 in the implant 4. The through bore 10 in the spacer element 9 is of a bore diameter 11 which is larger than an outside diameter 12 of the screw bolt 8 (see also FIG. 2b and FIG. 4). As a result, the screw bolt 8 is not in engagement with the spacer element 9 during screwing of the fixing screw 7 and the spacer element 9 does not turn with the fixing screw 7. In addition, in the illustrated example a screw head surface 16 of the screw head 15 of the fixing screw 7, that faces towards the film 6, is provided with a friction-reducing coating, for example with a Teflon coating. In that way it is possible to prevent the film 6 from also turning during the positionally stable fixing thereof by being clamped between the screw head 15 and the spacer element 9, whereby as a further consequence no unwanted folding of the film 6 in the clamping region occurs. Alternatively or additionally a friction-reducing coating can also be provided at a film surface 17.

FIG. 6 shows the dental implant system 1 of FIG. 5 after screwing of the fixing screw 7 to the implant 4. The spacer element 9 provided can achieve uniform coverage of the bone defect site 2 with the film 6, without for example unwanted folding or cratering occurring in the film 6. In addition to the film 6 being clamped between the screw head 15 and the spacer element 9 the film is anchored to the jawbone 3 by means of suitable fixing devices 40. The fixing devices 40 can involve for example metal or resorbable nails, pins or screws which are fixed through the film 6 to the jawbone 3. Alternatively the film 6 can also be glued to the jawbone 3.

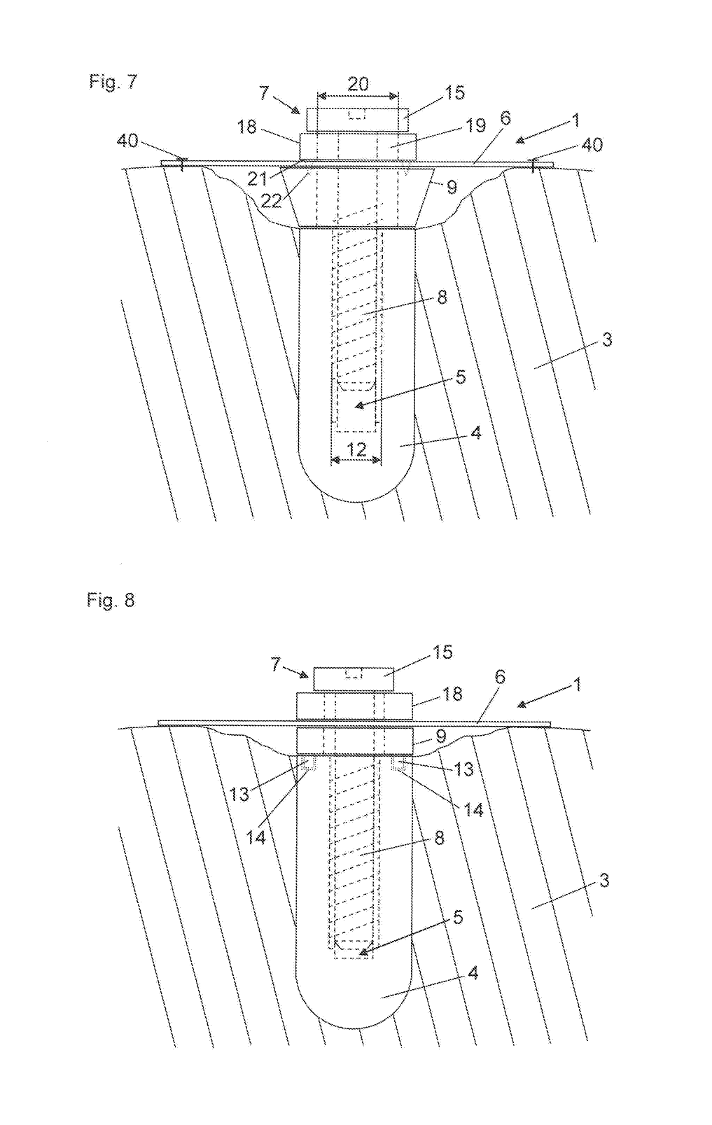

FIG. 7 shows a further example of a proposed dental implant system 1 in a sectional view through a jawbone 3. In this example the spacer element 9 has a conical or frustoconical peripheral surface, which provides an enlarged contact surface for the film 6 and which can simulate the anatomical root shape. In addition there is a fixing sleeve 18 which holds open a passage through the gum to the oral cavity and which has a through hole 19 of a hole diameter 20 larger than an outside diameter 12 of the screw bolt 8 of the fixing screw 7. As a result the male thread of the screw bolt 8 is not in engagement with the fixing sleeve 18 during screwing of the fixing screw 7 whereby the fixing sleeve 18 does not also rotate with the fixing screw 7. In that way it is possible to prevent the film 6 from also turning while it is being fixed in a stable position by clamping between the fixing sleeve 18 and the spacer element 9 whereby as a further consequence unwanted folding of the film 6 in the clamping region does not occur. At a sleeve surface 21 of the fixing sleeve 18, that is towards the film 6, there are also thorn-like extensions 22 which pass through the film 6 and thus represent a further rotation-preventing means for the film 6.

FIG. 8 shows a further embodiment of a proposed dental implant system 1 with a spacer element 9, a fixing sleeve 18 and a film 6 clamped between the spacer element 9 and the fixing sleeve 18. In this example the spacer element 9 is arranged on the implant 4 in rotationally locked relationship, insofar as projections 13 protruding from the spacer element 9 engage into corresponding recesses 14 on the implant 4. In this example the film 6 is glued at its edge regions to the jawbone 3.

FIG. 9 shows a jawbone 3 with anchored implant 4 after bone regeneration of the jawbone 3 in the cavity 27. After bone regeneration has occurred the fixing screw 7, the spacer element 9 and optionally the film 6 and the fixing sleeve 18 are removed and a pass-through post 29 is arranged on the implant 4, which projects into an oral cavity to a position above the gum 31 and to which a dental prosthesis 30 can be fixed. FIG. 10 shows an exploded perspective view of a proposed pre-bonded multi-layer film 6. The film 6 includes a forming layer 33 and two cover layers 34a and 34b. The forming layer 33 is stiffer than the cover layers 34a and 34b and has a shape structure 35. The shape structure 35 includes a plurality of strut-shaped forming elements 37 which serve to form the film 6 over a bone defect site 2 (this is not shown here), wherein the film 6 can be well shaped by the forming elements 37 to a jawbone 3 which is still present at the bone defect site 2. The shape structure 35 is overall of a substantially grid-shaped configuration and thus permits the provision of any surface shapes for the film 6 so that, in conjunction with a bone defect site 2, it is possible to form any desired cavity shapes between the film 6 and the bone defect site 2.

The forming layer 33 and the cover layers 34a and 34b respectively comprise a bioresorbable material so that the film 6 as a whole is substantially complete resorbable in the body. By virtue of the provision of two cover layers 34a and 34b, between which the forming layer 33 is embedded, it is possible to control in particular the resorption speed and mechanical strength of the forming layer 33.

The cover layers 34a and 34b can be for example bioresorbable collagen membranes which on the one hand by virtue of their softness can well cover a bone defect site 2 and which on the other hand can be well glued to a gum 31 surrounding the bone defect site 2 so as to afford good sealing for the bone defect site 2.

The forming layer 33 can for example comprise a bioresorbable polymer material or copolymer material. In particular the forming layer 33 can include for example about 82% L-lactic acid and about 18% glycolic acid. Such a choice of material affords a forming layer 33 which is substantially stable in shape and which is adapted to be mechanically, thermally and/or chemically deformable for shaping to a bone defect site 2, wherein after such deformation the forming layer 33 is substantially stable in shape again. By virtue of the stiffness and stability in respect of shape of the forming layer 33 therefore a cavity 27 for bone regeneration can be created between the film 6 and a bone defect site 2 and also maintained for the period of bone regeneration.

FIG. 11 shows a side view of the pre-bonded multi-layer film 6 as shown in FIG. 10.

FIG. 12 shows a plan view of a further variant of the proposed film 6 which in this example is of a double-layer nature and includes a forming layer 33 and a cover layer 34. Both the forming layer 33 and also the cover layer 34 are substantially flat. The film 6 can be cut to size as desired in order, depending on the respective situation of use, to permit good shaping to a bone defect site 2.

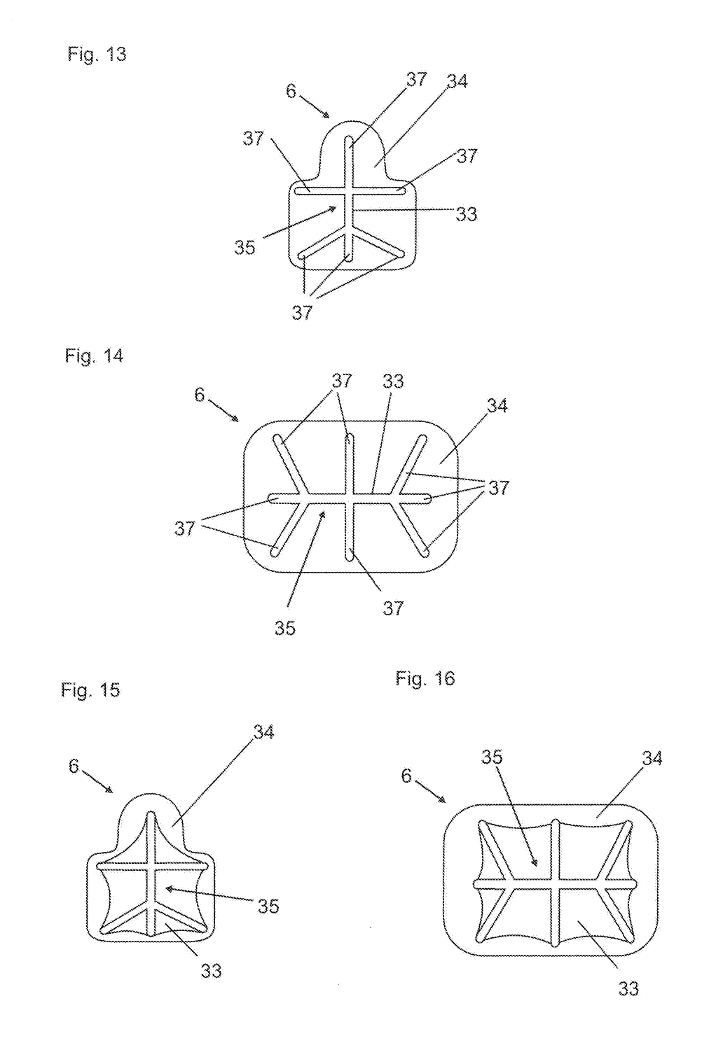

FIGS. 13 and 14 show two further embodiments of a proposed two-layer film 6 with different outside contours in respect of the cover layer 34 and different shape structures 35 of the forming layer 33.

FIGS. 15 and 16 show further example of proposed films 6, wherein, in the examples shown here, the forming layer 33 was respectively applied in the form of a gel to the cover layer 34 and subsequently hardened. The forming layers 33 shown here each include a shape structure 35 which for example was achieved by more gel being applied in the regions of the structure 35 so that the forming layers 33 are of differing layer thicknesses. In the region of a forming structure 35 a forming layer 33 is of a respectively greater layer thickness than in the other regions of the forming layer 33.

FIGS. 17 through 24 each show an exploded perspective view of further respective embodiments of a proposed film 6. The side 39 of a film 6, that faces downwardly in the respective Figures, is in this case the side 39 of the film 6, that is to face towards a bone defect site 2.

The examples of FIG. 17 and FIG. 18 are of a two-layer structure and respectively include a forming layer 33 and a cover layer 34, the forming layer 33 occupying a smaller surface area than the cover layer 34. The examples in FIG. 19 and FIG. 20 are of a three-layer structure and, besides a forming layer 33 and a cover layer 34, respectively include a carrier layer 38 to which substances like for example drugs, growth factors and/or other substances for protecting and promoting healing and bone formation can be applied.

The examples of FIG. 21 through FIG. 24 each have a forming layer 33 and two cover layers 34a and 34b, the forming layer 33 occupying a smaller surface area than the cover layers 34a and 34b. The examples of FIGS. 22 through 24 each additionally have a carrier layer 38 which can be provided with suitable substances (as described above in relation to FIG. 19 and FIG. 20).

1. A dental implant system for bone regeneration of a bone defect site of a jawbone including
an implant which is to be anchored in the jawbone and has a threaded bore,
a film for covering over the bone defect site and the implant, and
a fixing screw for positionally stable fixing of the film relative to the implant, wherein in the fitted position a screw bolt of the fixing screw projects through the film and can be screwed to the threaded bore in the implant,
wherein a spacer element can be arranged between the implant and the film, wherein in the fitted position the screw bolt projects through the spacer element,
wherein the dental implant system includes a substantially tubular fixing sleeve through which the screw bolt projects in the fitted position, wherein the film can be clamped between the spacer element and the fixing sleeve.

2. A dental implant system as set forth in claim 1, wherein the spacer element for passing the screw bolt therethrough has a through bore of a bore diameter larger than an outside diameter of the screw bolt.

3. A dental implant system as set forth in claim 1, wherein the spacer element can be arranged in rotationally locked relationship on the implant.

4. A dental implant system as set forth in claim 3, wherein provided as rotational locking means on the spacer element are projections which can be inserted into corresponding recesses of the implant.

5. A dental implant system as set forth in claim 1, wherein the spacer element is of a substantially hollow cylindrical shape, preferably in the form of a substantially tubular sleeve.

6. A dental implant system as set forth in claim 1, wherein the spacer element is of a substantially conical or frustoconical configuration.

7. A dental implant system as set forth in claim 1, wherein the fixing screw has a screw head, wherein the film can be clamped between the spacer element and the screw head.

8. A dental implant system as set forth in claim 7, wherein at a screw head surface which in the fitted position is towards the film the screw head is provided with a friction-reducing coating, preferably with a Teflon coating.

9. A dental implant system as set forth in claim 7, wherein the film is provided with a friction-reducing coating, preferably with a Teflon coating, at a film surface that is towards the screw head in the fitted position, at least in a contact region with the screw head.

10. A dental implant system as set forth in claim 1, wherein the fixing sleeve for passing the screw bolt therethrough has a through hole of a hole diameter which is larger than an outside diameter of the screw bolt.

11. A dental implant system as set forth in claim 1, wherein provided on the fixing sleeve at a sleeve surface which in the fitted position is towards the film is at least one extension preferably of a substantially thorn-like configuration—for positional fixing of the film relative to the fixing sleeve.

12. A dental implant system as set forth in claim 1, wherein the fixing sleeve is formed as a separate part

13. A dental implant system as set forth in claim 1, wherein the film is substantially completely resorbable.

14. A dental implant system as set forth in claim 1, wherein the film is a preferably pre-bonded multi-layer film which includes a shaping forming layer for shaping the film to the bone defect site and at least one cover layer for covering the bone defect site, wherein the forming layer and the at least one cover layer are substantially completely resorbable.

15. A dental implant system as set forth in claim 14, wherein the forming layer is stiffer than the at least one cover layer.

16. A dental implant system as set forth in claim 14, wherein the at least one cover layer at least partially and preferably substantially completely comprises a bioresorbable collagen material, wherein preferably the bioresorbable collagen material includes type-I-collagen and/or type-III-collagen.

17. A dental implant system as set forth in claim 14, wherein the forming layer at least partially and preferably substantially completely comprises a bioresorbable polymer material, wherein preferably the bioresorbable polymer material includes lactic acid, preferably L-lactic acid, and/or derivatives thereof.
